(19) United States
(12) Patent Application Publication
(10) Pub. No.: US 2011/0230807 A1
(43) Pub. Date: Sep. 22, 2011

(54) DEVICE FOR STABILIZING BODY SEGMENTS

(76) Inventor: Frank Friedrich Groehninger, Oberbexbach (DE)

(21) Appl. No.: 12/998,166

(22) PCT Filed: Sep. 23, 2009
(86) PCT No.: PCT/DE2009/075051

§ 371 Date: Mar. 24, 2011

(30) Foreign Application Priority Data
Sep. 26, 2008 (DE) 20 2008 012 828.5

(51) Int. Cl. A61F 5/00

(52) U.S. Cl. 602/13; 602/16

(57) ABSTRACT

The invention relates to a device for stabilizing body segments. In order to provide a novel device for stabilizing body segments, particularly for use in the area of sports and rehabilitation medicine, that can be sold as a prefabricated, ready-made item or as a semi-finished, tailor-made item, the invention proposes to engineer an orthotic device that makes anatomically congruent contact with the respective body segments, a textile substrate having the shape of the body segment to be stabilized has an anatomically congruent layer of plastic applied thereon that stabilizes the shape of the textile substrate, a bendable metal element being surrounded by the plastic layer, the device being designed to cover one or more additional body segments adjacent to the body segment in question as well as the joint(s) therebetween, and means to restrict the angle of movement of the joint(s) being provided. In this manner, a device for stabilizing body segments can be provided which is of low weight and offers the wearer a high degree of comfort, because the textile substrate faces the wearer's skin. This device may be used to fully or partially restrict, stabilize or support movements of the various joints, and may thus also be used to enhance the wearer's natural biodynamics. Thanks to the anatomically congruent plastic layer, natural, concave parts of the body's anatomy, for example in the area of the lumbar lordosis, need not be “bridged” as with conventional bandages, and it is possible to apply individually adjustable pressure to designated areas of the body.

The invention relates to a device for stabilizing body segments.

The U.S. Pat. No. 6,779,282 B2 discloses an athletic sock having an insole extending from the toes to the heel and including a stirrup-shaped device in the form of two interconnected uprights of suitable material connected with each other in order to stabilize the ankle, and a circular strap running at right angles to the uprights and serving to restrict movement of the talocalcaneonavicular joint and stabilize the ankle. According to this invention, the insole is bonded to the athletic sock, for example by means of a plastic spray-bonding method. A plastic layer may also be provided on the two uprights. Use of this device makes it unnecessary to stabilize the joint by means of taping.

Plaster bandages for immobilizing body segments are also known. These are used mainly in the medical field, especially for bone fractures. Such bandages, however, have to be applied individually, which is time-consuming and costly.

Plastic braces, too, are known, which are likewise used in the field of medicine. They are being increasingly preferred to plaster bandages because they are lighter in weight.

The object of this invention is to provide a novel device for stabilizing body segments, which is particularly intended for use in the field of sports and rehabilitation medicine and which can be sold as a pre-fabricated, ready-made item or as a semi-finished, tailor-made item.

This object is established according to the invention in that a textile substrate having the shape of the body segment to be stabilized has a shape-stabilizing plastic layer applied thereon.

According to the invention, a functional orthotic device is provided that makes anatomically congruent contact with the respective body segments. Whereas bandages currently available on the market have to bridge natural, concave parts of the body (for example in the area of the lumbar lordosis), thus necessitating the use of a pelotte to exert pressure, the device according to the invention follows the body contours in all areas, including concave areas. Thus a snugger fit is obtained, and undesirable pressure exerted by pelottes or the like on areas of the body is avoided. On the contrary, a case-by-case decision can be made as to where pressure should be exerted on the body and where not. In this way, a device for stabilizing body segments is provided which may be referred to as an orthotic bandage or a bandage-type orthosis, and which offers low weight combined with high wearing comfort because the textile substrate faces the user's skin. This device can be used to fully or partially restrict, stabilize or support movements of the various joints, and can thus serve to enhance the wearer's natural biodynamics.

The stabilizing plastic layer may be net-like or planar, flexible or rigid, elastic, hard or soft, two- or three-dimensional, with the additional possibility of combining a plurality of these properties with each other in one device, for example by making selective use of different plastic layers for different areas of the device, for instance to support a certain joint and to only partially support another one. The plastic layer can furthermore be configured as a single layer or a multi-layer. The plastic layer is immovable on account of its being bonded to the textile substrate. It may be sprayed onto the textile substrate fabric both outside and inside, and covered with a slip aid. Of course it is possible to provide additional, soft plastic applications, for example made of silicone, as pelottes (pressure pads) on the device in order to selectively exert pressure on certain parts of the body.

Body segments are defined according to the invention as the upper and lower limbs or the trunk.

It is within the scope of the invention that the textile substrate is a knitted or woven textile fabric.

Generally, both alternatives are possible. The textile substrate may also be elastic, for example in the form of an elastic knitted or woven textile fabric, a rubber-backed knitted textile fabric, or a combination thereof Structures resembling textiles, for example neoprene, are generally also possible.

A refinement of the invention consists in that the device is made up of several layers.

The device may be engineered to be rigid, elastic or flexible.

It is possible to configure the device not only as a single-layer device but also as a multi-layer device.

An embodiment of the invention consists in that the device covers one or more additional body segments adjacent to the body segment in question as well as the joint(s) therebetween.

Obviously, not only the adjacent joint is covered by the device, but, at least partially, also the other body segment adjacent to this joint. In the case of a device intended to stabilize the upper part of the body and an arm, at least some of the upper part of the body would be covered, as well as the shoulder joint and part of the humerus.

In this connection it is possible to provide means to restrict the angle of movement of the joint(s).

It is also within the scope of the invention to provide at least one stabilizing strap to enhance adhesion.

A stabilizing strap of such kind may be configured to provide statically rigid or elastic anchorage, or anchorage comprising a combination of these two properties.

A refinement of the invention consists in that an insert, in particular of metal or plastic, is surrounded by the plastic layer.

An insert of such kind may, for example, consist of a bendable metal element that makes it possible, after the actual production process, to individually shape the still mouldable device to fit a body part, or of a metal element in the shape of a body part.

It is furthermore within the scope of the invention to provide, on the inside of the device, i.e. on the side facing the user's body, a pressure chamber that can be filled with air via a valve.

The device can be made to fit even more snugly against the body by pumping up a pressure chamber of this kind, while swellings can be compensated by releasing air; pressure may also be exerted on stabilized body segments via the pressure chamber.

An embodiment of the invention is described below by referende to the drawings.

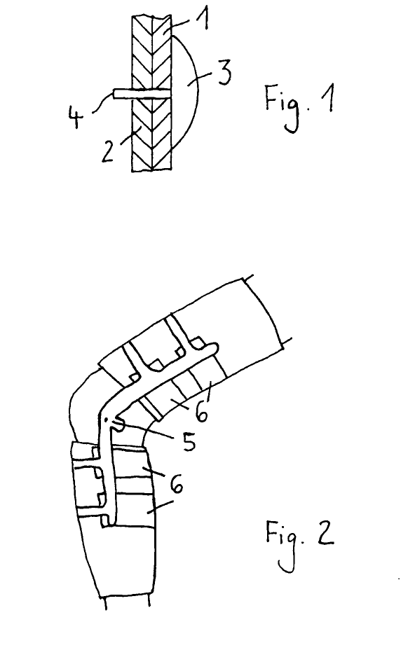

FIG. 1 is a schematic diagram showing the make-up of the device;

FIG. 2 is a schematic diagram of a device according to the invention for stabilizing the thigh and lower leg and the knee joint connected therebetween during mediolateral (M-L), anterior-posterior (A-P) and rotational movement.

As is evident from FIG. 1, the device according to the invention has a substrate 1 tailored to match the shape of the body segment(s) concerned. The substrate 1 may be a knitted textile fabric or a woven textile fabric. This textile substrate 1 has a plastic layer 2 applied thereon which stabilizes the shape of the textile substrate 1. Provision may be made to repeat this layering, like a sandwich, in certain highly-stressed areas or over the entire device until the desired mechanical loading capacity has been reached.

The device may also feature a pressure chamber 3 that can be inflated with air to make the device fit more snugly against the particular body segment. Air can likewise be released from the chamber while the device is in place in order to somewhat loosen the fit against the body segment. This pressure chamber 3 has a valve 4 via which the pressure chamber 3 may be inflated or deflated.

The device illustrated also has a means 5 for restricting the knee's angle of movement. Said means is configured as a joint 5 that is connected to both parts of the device, being located between the part of the device embracing the thigh and the part embracing the lower leg, and that has an adjustable maximum bending angle. The anchoring straps 6 may be attached horizontally or in any other position.

1. Device for stabilizing body segments, wherein, to engineer an orthotic device that makes anatomically congruent contact with the respective body segments, a textile substrate (1) having the shape of the body segment to be stabilized has an anatomically congruent layer of plastic (2) applied thereon that stabilizes the shape of the textile substrate, a bendable metal element being surrounded by the plastic layer (2), the device being designed to cover one or more additional body segments adjacent to the body segment in question as well as the joint(s) therebetween, and means (5) to restrict the angle of movement of the joint(s) being provided.

2. Device according to claim 1, wherein body segments are the upper and lower limbs or the trunk.

3. Device according to claim 1, wherein the textile substrate (1) is a knitted textile fabric or a woven textile fabric.

4. Device according to claim 1, wherein the device is made up of a plurality of layers.

5-6. (canceled)

7. Device according to claim 1, wherein at least one stabilizing strap is provided to enhance adhesion.

8. (canceled)

9. Device according to claim 1, that wherein on the inside of the device, facing the wearer's body, a pressure chamber (3) is provided which can be inflated with air via a valve.
